(19) United States
(12) Patent Application Publication
(10) Pub. No.: US 2005/0256454 A1
(43) Pub. Date: Nov. 17, 2005

(54) Device for clamping large blood vessels

(76) Inventor: Mariangela Moggi, Modena (IT)

Correspondence Address: YOUNG & THOMPSON, 745 SOUTH 23RD STREET, 2ND FLOOR, ARLINGTON, VA, 22202, US

(21) Appl. No.: 10/515,992

(22) PCT Filed: May 29, 2003
(86) PCT No.: PCT/IT03/00335

§ 371 Date: Nov. 30, 2004

(30) Foreign Application Priority Data
Jun. 5, 2002 (IT) MI02002A000152

(51) Int. Cl. A61M 29/00

(52) U.S. Cl. 604/102.01; 604/101.05

(57) ABSTRACT

The device includes: a first catheter having dilators, a first inflatable balloon active between an inflated configuration, in which the first inflatable balloon is filled with a pressurised fluid and extends in a radial direction away from the first catheter, and a deflated configuration; elements for sending the fluid to the first inflatable balloon; an external tube, axially slidable on the first catheter, for covering and uncovering the first inflatable balloon in the deflated configuration; a second catheter internally insertable in the first catheter and having an internal passage for a metal guide wire; a second inflatable balloon active between an inflated configuration, in which the second inflatable balloon is filled with a pressurised fluid and extends in a radial direction away from the second catheter, and a deflated configuration; elements for sending fluid to the second inflatable balloon.

### TECHNICAL FIELD

The invention is usefully, though not exclusively, applied in the field of aortic and iliac clamps.

### BACKGROUND ART

The typical pathology involving the use of the present invention is abdominal aortic aneurysm. An aneurysm is a dilation of an arterial wall caused by blood pressure. The most frequent cause of the pathology is arteriosclerosis, and the most common location is the artery located in the abdomen downstream of the renal arteries.

The abdominal aortic aneurysm most commonly affects older people and has a slow or slowish evolution towards rupture. When rupture occurs, acute haemorrhaging follows which, unless there is swift intervention by experts, rapidly leads to death. Even where the intervention is swift, death often occurs. Vascular technology and better non-invasive diagnostic methods (for example using ultra-sound or CT scanning) have led to improvements in diagnosis and to the discovery that the pathology is gradually affecting more, usually older people; there has been an according increase in preventive operations, though between 20% and 50% of interventions are still performed as a matter of urgency.

The surgical solution to abdominal aortic aneurysm consists in applying clamps upstream and downstream of the dilated arterial tract, usually downstream of the renal arteries and upstream of the iliac arteries, and suturing a stent-graft made of synthetic material (dacron or PTFE) into the healthy parts of the aorta, from one end to the other of the dilated tract. In cases where the dilation extends into the two branches into which the aorta is sub-divided, the stent-graft used is bifurcated and must be sutured at a more downstream point.

This technique, very effective if carried out before aneurysm, is very disappointing however when used after rupture has occurred. The difficulties are connected with the need to proceed to a very invasive intervention, requiring a large laparotomy in patients who are under the effects of shock, where even anaesthesia or initial laparotomy alone can lead to very serious blood pressure drops, with the distinct possibility of further serious complications. It is often not easy to isolate the vessels involved in the haemorrhage and the preliminary stages of clamping themselves can lead to haemorrhaging. It is also true that in very many cases the patient admitted in emergency conditions has not been subjected to preliminary angiograpliic examinations and as a result there are no indications of an anatomical nature as to where the vessels in need of treatment are located. In emergency cases, the possibility of obtaining haemostasis before anaesthesia and laparotomy, and therefore removing the ruptured tract from circulation, would enable the stages of diagnosis and surgery to be performed much more methodically and efficiently, and would also enable endoprostheses to be applied with considerably reduced invasiveness when locating clamp points.

The main aim of the present invention is to provide an instrument which, using a percutaneous technique, guarantees a rapid halt to arterial haemorrhaging, or at least which will lead to isolation of easily-accessed vessels such as the femoral arteries.

These aims and more besides, which will better emerge from the description that follows, are all achieved by a clamping device for large vessels, as it is characterised in the appended claims.

### DISCLOSURE OF THE INVENTION

Further characteristics and advantages of the present invention will better emerge from the detailed description that follows of the clamping device of the invention, illustrated purely by way of non-limiting example in the accompanying figures of the drawings, in which:

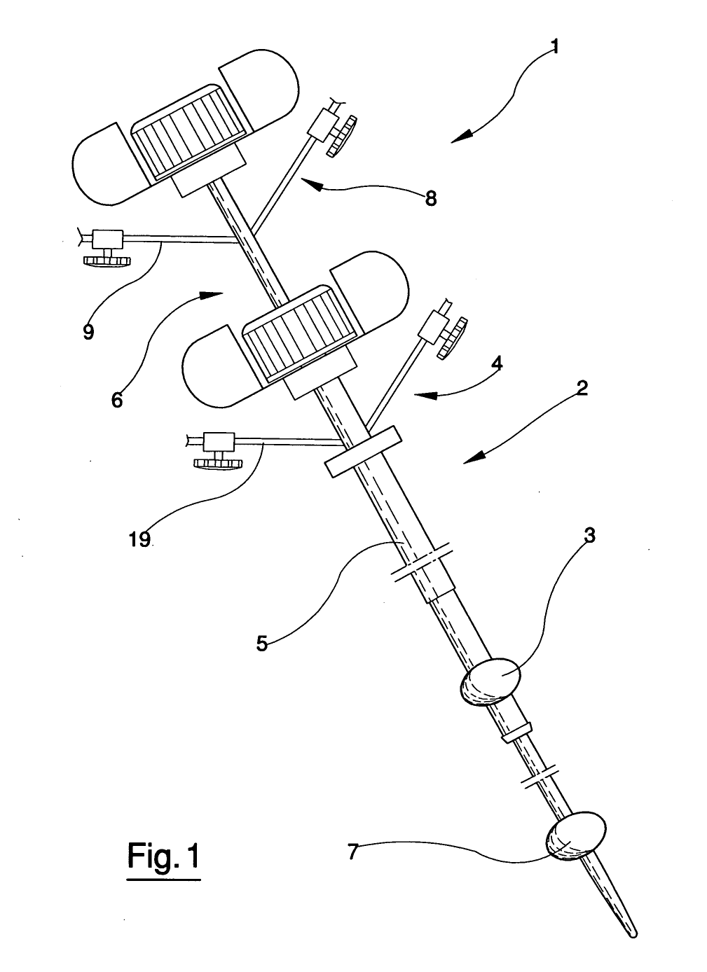

FIG. 1 is an overall view of the device of the present invention;

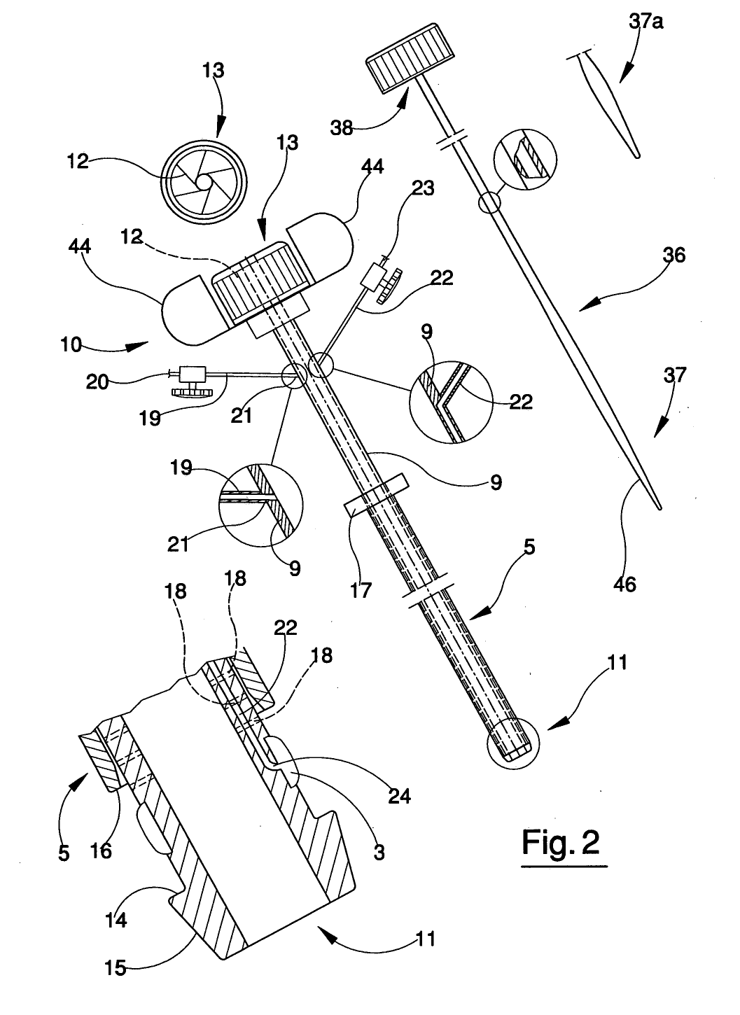

FIG. 2 is a first view, in greater detail, of the device of the invention;

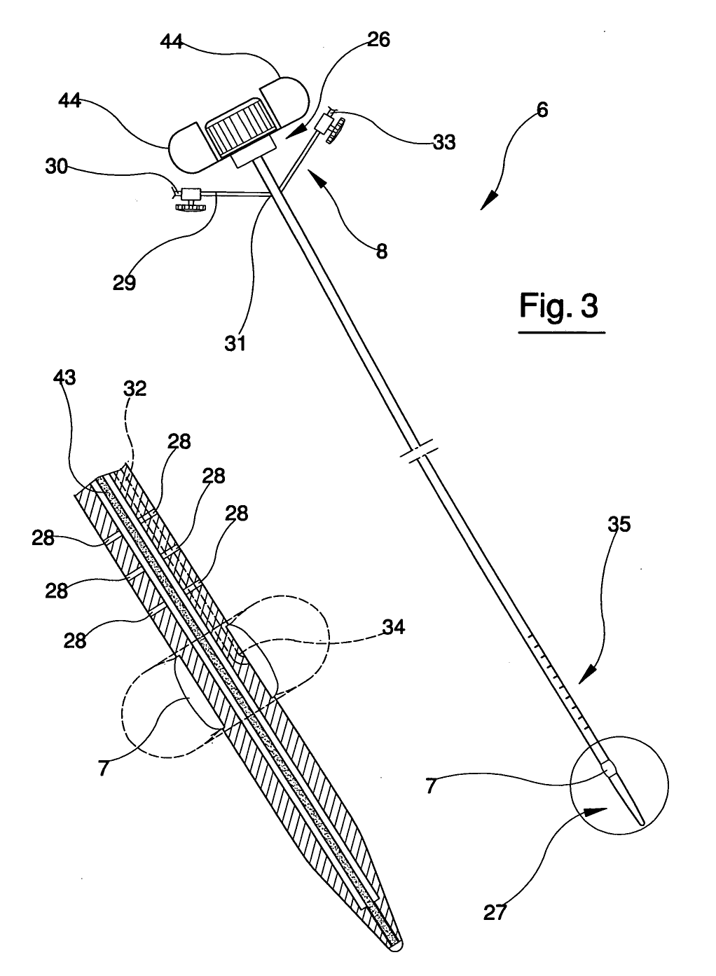

FIG. 3 is a second view in greater detail of the device of the invention;

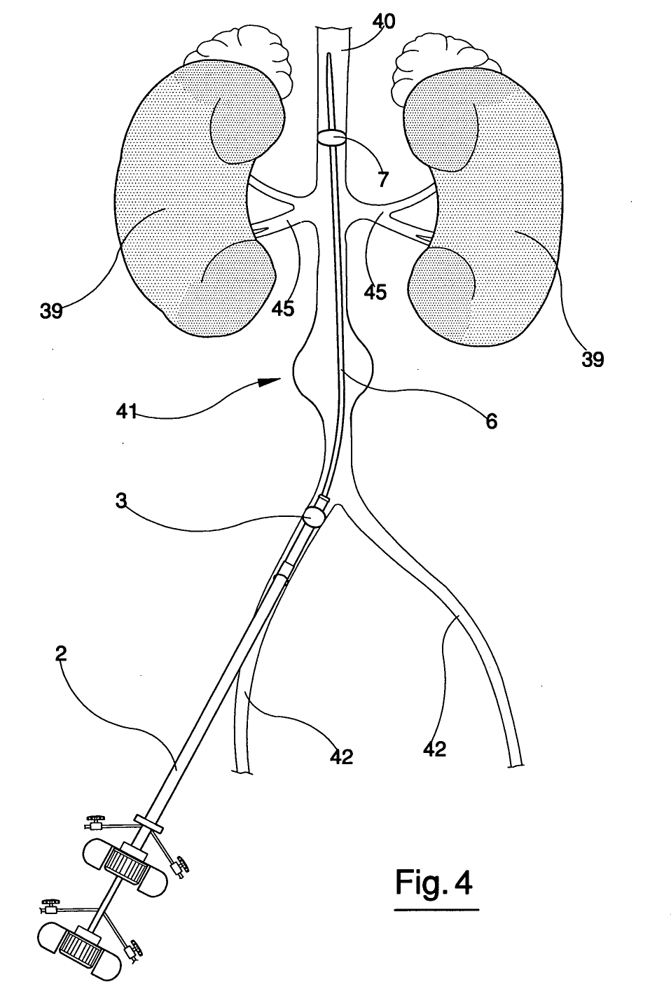

FIG. 4 is a view of the device of the present invention in use.

With reference to FIG. 1, the device of the invention is denoted in its entirety by 1.

The device comprises a first catheter 2, to which a first inflatable balloon 3 is attached, which can have an inflated configuration, in which the balloon is filled with a pressurised fluid and extends radially away from the first catheter 2, and a deflated configuration. The first catheter 2 further comprises means 4 for sending the fluid to the first inflatable balloon, and an external tube 5, axially slidable on the first catheter 2, for covering and uncovering the first inflatable balloon 3 when in the non-inflated configuration.

The device of the invention further comprises a second catheter 6, insertable internally of the first catheter 2. The second catheter 6 is associated to a second inflatable balloon 7, active between an inflated configuration, in which the balloon is filled with a pressurised fluid, and a deflated configuration. The second catheter 6 further comprises means for sending the fluid to the second inflatable balloon 7.

The first catheter 2 essentially comprises a first internal tube 9 having a first end 10 and a second end 11, in which the second end exhibits the first inflatable balloon 3. A screw-operated haemostatic valve 12 in internally engaged to the first end 10 of the first internal tube 9 for arresting any flows of blood through the first tube 9. The first catheter 2 further comprises means 13 for axially blocking the second catheter 6 internally of the first catheter 2, for example self-centring jaws constituting a sort of a chuck, arranged internally of the haemostatic valve 12 and actionable by means of the same screwing manoeuvre which operates the haemostatic valve 12.

The first internal tube 9 exhibits, in proximity of the second end 11 thereof, an external radial increase in section, defining an annular surface 14 which is substantially perpendicular to the longitudinal development of the first internal tube 9 and faces the first end 10. The radial increase in section decreases in the direction of the second end 11 of the first internal tube 9, defining a tapered external surface 15. The second end 11 of the first internal tube 9 exhibits a trunco-conical shape, developing externally, which facilitates insertion thereof in an artery, as will be more clearly described herein below. The first inflatable balloon 3 is arranged in proximity of the annular surface 14, before the tapered surface 15.

The external tube 5 which is coaxially slidable on the first catheter 2 exhibits a first end 16 and a second end 17. The external tube 5 is arranged coaxially to the first internal tube 9 and is externally slidable thereon between a first position in which the first end 16 of the external tube is in contact with the annular surface 14 and the external tube covers the first inflatable balloon 3, and a second position in which the first end 16 of the external tube 5 is distanced from the annular surface 14 and the external tube 5 does not cover the first inflatable balloon 3. When the external tube 5 is in die first position, i.e. with the first end thereof striking on the annular surface 14, the first catheter exhibits, at least for a certain tract, an external surface which is practically without sharp points and which is tapered to an end at the tapered surface 15. The first internal tube 9 can further exhibit a plurality of radial holes 18 arranged in proximity of the annular surface 14 and on the opposite side to the tapered surface 15. The radial holes 18 can for example send liquid into the zone downstream of the first inflatable balloon 3 through a proximal access 19 (this manoeuvre can be performed by occluding the end 11 with a special dilator), and can be covered or uncovered according to the direction flow of the external tube 5. When the holes are covered, aspiration can be performed through the first catheter 2 using the same proximal access 19.

The first internal tube 9 further comprises a lateral conduit 19 having free first end 20, and a second end 21 which is connected in proximity of the first end 10 of the first internal tube 9 and is in fluid communication with the inside of the first internal tube 9. The lateral conduit 19 can for example be used to send or aspirate liquids internally of the first catheter 2. Any liquids introduced into the lateral conduit 19 can for example exit from the first catheter 2 through the radial holes 18, and any liquids which have penetrated into the first catheter 2 through the radial holes 18 can be aspirated along the lateral conduit 19.

The means 4 for sending the fluid to the first inflatable balloon 3 comprise a conduit 22 afforded in the first internal tube 9 and having a first outlet 23 arranged externally and in proximity of the first end of the first internal tube 9 and a second outlet 24 in fluid communication with the inside of the first inflatable balloon 3. It is possible to send, for example, air through the conduit 22 into the first inflatable balloon 3 in order to inflate the balloon 3, and it is, vice-versa, possible to deflate the first inflatable balloon 3 by opening the first outlet 23.

The second catheter 6 comprises a second internal tube 25 having a first end 26 and a second end 27. The second inflatable balloon 7 is arranged in proximity of the second end 27 of the second internal tube 25. The second internal tube 25 exhibits a plurality of radial through-holes 28 arranged in proximity of the second inflatable balloon 7, at the first end 26. The catheter internally includes a non-uniform channel running throughout its length for a metal guide-wire.

The second internal tube 25 further comprises a lateral conduit 29, having a free first end 30 and a second end 31 which is connected in proximity of the first end of the second internal tube 25 and which is in fluid communication with the inside of the second internal tube 25. The function of the lateral conduit 29 and the radial through-holes 28 is the same as the function of the lateral conduit 19 and the radial through-holes 18 described herein above. The lateral conduit 29 can be used to send or aspirate liquids inside the second catheter 6. Any liquid introduced in to the lateral conduit 29 can exit from the second catheter 6 through the radial holes 28, and it is also possible to aspirate along the lateral conduit 29 any liquid which has penetrated into the second catheter 6 through the radial holes 28.

The means 8 for sending the fluid to the second balloon 7 comprise a conduit 32, afforded in the wall of the second internal tube 25 and having a first exit 33 arranged externally of the second internal tube 25 in proximity of the first end 26 and a second exit 34 in fluid communication with the inside of the second inflatable balloon 7. The conduit 32 performs the same function for the second inflatable balloon 7 as the conduit 22 performs for the first balloon 3; it is possible to send air or liquids through the conduit 32 to inflate the second inflatable balloon 7, or to open the first exit 33 and deflate the second inflatable balloon 7.

The second catheter 6 also exhibits a plurality of radio-opaque markers 35 arranged so as to define a measurement scale. The incisions can be useful in evaluating the dimensions of a blood-vessel aneurysm, as will be better described herein below.

The device of the present invention further comprises a dilator 36 having an elongate axial symmetric shape with a first end 37 being tapered and defining a wedge-shaped surface 46, and a second end 38. The dilator 36 is slidable internally of the first catheter 2 and can assume a position in which the first end 37 projects out of the second end 11 of the first internal tube 9. When the dilator 36 is inserted in the first catheter 2 and the first end 37 projects from the second end 11 of the first internal tube 9, the wedge-shaped surface 46 connects to the tapered external surface 15 located on the second end 11 of the first internal tube 9. In this configuration the insertion of the first catheter 2 in a blood vessel is very much simplified. The dilator 36 has an internal conduit for insertion of a metal guide wire.

The dilator 36 can have a calibre at its apex of such dimensions as to occlude the first catheter 2 at the end 11; while in its other parts it can be of a smaller calibre and thus enable passage of liquids up to the radial holes 18.

With reference to FIG. 4, the operation of the device of the invention for treatment of abdominal aneurysm can be described as follows.

Number 39 denotes the kidneys, 40 the abdominal aorta which bifurcates into the iliac-femoral arteries 42. The figure illustrates an aneurysm 41 in the tract of aorta comprised between the kidneys 39 and the bifurcation of the arteries 42. In a first stage the first catheter 2 is introduced, with a metal guide wire, in one of the femoral arteries 42. At this stage, the dilator 36, which is inserted in the first catheter 2, facilitates the sliding of the catheter 2 in the femoral artery. During the introduction, the external tube 5 contacts the annular surface 14 by an end 16 thereof, covering the first inflatable balloon 3 and further facilitating the sliding of the first catheter 2.

When the first inflatable balloon 3 has reached a position immediately downstream of the aorta bifurcation, the first balloon 3 is inflated, causing a block in arterial circulation in the iliac artery it has been inserted in. At this moment it can be useful to fix the catheter by suturing to the patient's skin, using slots 44 arranged on the first end 10 of the first internal tube 9.

In cases where the patient is in a state of shock, this manoeuvre can be delayed until after application of the second catheter 6 and inflation of the relative inflatable balloon 7 in the aorta upstream of the kidneys.

When the first balloon 3 has been inflated, the dilator 36 is removed from the first catheter 2. Blood flows internal of the first catheter 2 are prevented by the presence of the haemostatic valve 12, which enables introduction of the second catheter 6 internally of the first catheter 2. The second catheter 6 is made to slide through the first catheter 2 upstream of the bifurcation of the abdominal aorta up until the second inflatable balloon 7 reaches upstream of the renal arteries 45, where the second inflatable balloon 7 is inflated to block the arterial circulation throughout the downstream tract. The introduction of the second catheter 6 can be facilitated by the use of a guide wire 43 inserted in the second internal tube 25. The guide wire 43 also realizes the closure of the second end 27 of the second internal tube 25.

To enable injection of liquids through the radial holes 28 only, or to prevent back-flows from the second end 27, the conduit for the metal guide wire is of such dimensions as to enable the guide wire to fit snugly therein at the apex of the conduit and in the other parts to be of a slightly greater diameter.

When the second inflatable balloon 7 is inflated total haemostasis is achieved, including any haemorrhaging caused by the rupture of the aneurysm 41. Thus an accurate examination of the patient's condition can be carried out to decide which type of operation should be performed on the aneurysm.

As previously described, the classic intervention consists in suturing a synthetic stent-graft (dacron or PTFE) at healthy points of the aorta, which synthetic stent-graft leads from one end to the other of the dilated tract of artery. For correct assessment of the size and conformation of the stent-graft, an angiogram sending a dye to the dilated aortic tract through the radial holes 28 and comparing the dimensions of the dilation with the radio-opaque markers 35 located on the second catheter 6.

Having precisely defined the characteristics of the stent-graft to be used, a subsequent surgical intervention will require a much smaller laparotomy, as it is no longer necessary to identify the clamping sites for the aorta and one iliac. For the other iliac, contralateral endoluminal clamping can be performed using a simple catheter with an inflatable balloon, the first catheter 2 can be used.

An initial approach with application of guides and a first catheter 2 on both femorals should provide the best possible starting conditions for proceeding to the subsequent manoeuvres concerning the most favourable side in accordance with the anatomy of the aneurysm. If it is possible to use a contralateral approach and apply an endoprosthesis in combination with the aortic clamping, an endoprosthesis can be inserted by endolumninal introduction in the femoral artery not used for the introduction of the first and second catheter.

1). A device for clamping large blood vessels, comprising:
a first catheter (2);
a first inflatable balloon (3), associated to the first catheter (2) and active between an inflated configuration, in which the first inflatable balloon (3) is filled with a pressurised fluid and extends in a radial direction away from the first catheter (2), and a deflated configuration;
means (4) for sending the fluid to the first inflatable balloon (3);
an external tube (5), axially slidable on the first catheter (2), for covering and uncovering the first inflatable balloon (3) in the deflated configuration thereof;
a second catheter (6), which can be inserted internally of the first catheter (2);
a second inflatable balloon (7), associated to the second catheter (6) and active between an inflated configuration, in which the second inflatable balloon (7) is filled with a pressurised fluid and extends in a radial direction away from the second catheter (6), and a deflated configuration;
means (8) for sending the fluid to the second inflatable balloon (7).

2). The device of claim 1, wherein the first catheter (2) comprises a first internal tube (9) having a first end (10) and a second end (11), the second end (11) bearing the first inflatable balloon (3).

3). The device of claim 2, wherein the first catheter (2) further comprises a screw-operated haemostatic valve (12), internally engaged to the first end (10) of the first internal tube (9).

4). The device of claim 1, wherein the first catheter (2) comprises means (13) for axially blocking the second catheter (6) internally of the first catheter (2).

5). The device of claim 2, wherein the first internal tube (9) exhibits, in proximity of the second end (11), an external radial increase in section, defining an annular surface (14) which is perpendicular to the longitudinal development of the first internal tube (9) and which faces towards the first end (10).

6). The device of claim 5, wherein the radial increase decreases towards the second end (11) of the first internal tube (9), defining a tapered external surface (15).

7). The device of claim 6, wherein the external tube (5) exhibits a first end (16) and a second end (17), the external tube (5) being arranged coaxially to the first internal tube (9) and being externally slidable on the first internal tube (9) between a first position in which the first end (16) of the external tube (5) is in contact with the annular surface (14) and the external tube (5) covers the first inflatable balloon (3), and a second position in which the first end (16) of the external tube (5) is not in contact with the annular surface (14) and the external tube (5) does not cover the first inflatable balloon (3).

8). The device of claim 6, wherein the first internal tube (9) can exhibit a plurality of radial through-holes (18) arranged in proximity of the annular surface (14) and on an opposite side of the annular surface (14) to the tapered external surface (15).

9). The device of claim 7, wherein the first internal tube (9) comprises a lateral conduit (19) having a free first end (20), and a second end (21) which is connected in proximity of the first end (10) of the first internal tube (9) and which is in fluid communication with an inside of the first internal tube (9).

10). The device of claim 6, wherein the first inflatable balloon (3) is arranged in proximity of the annular surface (14) on an opposite side thereof with respect to the tapered external surface (15).

11). The device of claim 2, wherein the means (4) for sending fluid to the first inflatable balloon (3) comprise a conduit (22) for introducing fluid internally of the first inflatable balloon (3), which conduit (22) is afforded internally of the first internal tube (9) and which conduit (22) has a first outlet (23) arranged externally and in proximity of the first end (10) of the first internal tube (9) and a second outlet (24) in fluid communication with an inside of the first inflatable balloon (3).

12). The device of claim 1, wherein the second catheter (6) comprises a second internal tube (25) having a first end (26) and a second end (27).

13). The device of claim 12, wherein the second internal tube (25) exhibits a plurality of radial through-holes (28) arranged in proximity of the second inflatable balloon (7), on a side thereof nearer the first end (26).

14). The device of claim 12, wherein the second internal tube (25) comprises a lateral conduit (29), having a free first end (30) and a second end (31) which is connected in proximity of the first end (26) of the second internal tube (25) and which is in fluid communication with the inside of the second internal tube (25).

15). The device of claim 1, wherein the second catheter (6) exhibits a plurality of radio-opaque markers (35) arranged to define a measurement scale.

16). The device of claim 12, wherein the second inflatable balloon (7) is arranged in proximity of the second end (27) of the second internal tube (25).

17). The device of claim 12, wherein the means (8) for inflating the second inflatable balloon (7) comprise a conduit (32) for introduction of fluid internally of the second inflatable balloon (7), which conduit (32) is afforded in the second internal tube (25) and has a first exit (33) which is arranged externally of the second internal tube (25) in proximity of the first end (26) of the second internal tube (25), and a second exit (34) in fluid communication with the inside of the second inflatable balloon (7).

18). The device of claim 1, wherein the device comprises a dilator (36) having an internal passage for a metal guide wire, which internal passage has an elongate axial symmetric shape and a first end (37) and a second end (38), the first end (37) being tapered to define a wedge-shaped surface (46), the dilator (36) being slidable internally of the first catheter (2), and being able to assume a position in which the first end (37) of the dilator (36) projects externally of the second end (11) of the first internal tube (9).

19). The device of claim 18, wherein the dilator (36) can have at an apex thereof a calibre which is such as to occlude the first catheter (2) at the end (11) and a smaller calibre in other parts thereof in order to enable passage of liquids up to the radial through-holes (18).

20). The device of claim 12, wherein the second internal tube (25) is provided with an internal passage for a metal guide wire (43); a calibre of the internal passage being smaller at the second end (27) than at other parts thereof.
